(19) United States
(12) Patent Application Publication
(10) Pub. No.: US 2006/0211021 A1
(43) Pub. Date: Sep. 21, 2006

(54) Methods and compositions for evaluating cell function in sensory neurons

(76) Inventors: David C. Yeomans, Stanford, CA (US); Michael C. Peters, Hobart, IN (US); William F. Gilly, Pacific Grove, CA (US); Mary Peters, Hobart, IN (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/361,101

(22) Filed: Feb. 24, 2006

Related U.S. Application Data
(60) Provisional application No. 60/657,637, filed on Feb. 28, 2005.

(51) Int. Cl. C12Q 1/68; C12N 5/08; C12N 15/87

(52) U.S. Cl. 435/6; 435/455; 435/368

(57) ABSTRACT

Methods and compositions are provided for determining whether target, e.g., an endogenous or novel (non-endogenous), nucleic acid sequence of a neuronal cell is involved in sensory function. In practicing the subject methods, a neuronal cell selective vector including a modulating domain for a neuronal cell nucleic acid sequence, optionally having a domain encoding a directly detectable product, is administered to an animal. Sensory function in harvested neuronal cells is then evaluated to determine whether the target nucleic acid sequence is involved in sensory function. Also provided are compositions, kits, and systems for practicing the subject methods.

### CROSS-REFERENCE

This application claims the benefit of U.S. Provisional Application No. 60/657,637, filed Feb. 28, 2005, which application is incorporated herein by reference.

### INTRODUCTION

1. Background of the Invention

Pain exerts an enormous toll on society, costing an estimated $200 billion in medical charges and lost wages, and incalculable costs in terms of human suffering. Pain is detected by specialized neurons termed “nociceptors” which transduce painful stimuli and carry that information to the central nervous system by propagating electrical signals known as action potentials along their peripheral fibers or processes. These fibers are either unmyelinated (C fiber) or thinly myelinated (Aδ fibers); the cell bodies of both residing in the dorsal root ganglia (DRG) just outside the spinal cord. Depending on the nature of the painful event, a variety of responses are possible, ranging from rapid withdrawal from the source of stimulation to the development of long-term hyperalgesia in the area surrounding an injury. While most responses are clearly adaptive, others are not. In particular, a state of chronic pain occurs in a variety of situations and is a major clinical problem that has defied safe, effective solutions.

To move forward with drug development, the role of genes in the onset and advancement of pain must be determined. Currently, small animal models have been used to study pain transmission and signal conduction at an organismal level, as well as to evaluate the role of neuronal cell genes in such actions. Although transgenic mice are experimentally accessible animal models of disease, they require the expenditure of a large quantity of time and labor for their production. Therefore, the requirements for conventional transgenic technologies have hampered their own usefulness.

Similarly, “gene knockout” models also pose the same problems. Although the “gene knockout” mouse is the standard for a loss-of-function model, inactivating more than a few candidate genes is currently a daunting proposition. In addition to the problems of time and expense, knockout or transgenic animal methods produce models with a genetic change throughout the entire animal, which might be fatal, or at least may affect systems other then the target system. Therefore, the development of a new system for characterizing the role of neuronal cell genes in signal conduction is needed.

There is a continued need in the field for new methods of creating animal models to evaluate the role of an endogenous nucleic acid sequences in sensory neurons. The present invention solves this need.

2. Relevant Literature

U.S. patent of interest include: U.S. Pat. Nos. 6,383,738; 5,849,572; and 5,849,571. Published U.S. Applications of interest include: 20020155432; and 20020098168. Additional references of interest include: Wilson & Yeomans, Curr. Rev. Pain (2000) 4(6):445-50; Finegold et al. Hum. Gene Therapy (1999) 10(7):1251-1257; Pihl at al. Eur. J. Pharmacology (2001) 429(1-3):39-48; Wilson et al. Proc. Nat'l Acad. Sci. USA (1999) 96(6):3211-3216; Yao et al. Gene Therapy (2003) 10(16)1392-1399; Goss et al. Gene Therapy (2001) 8(7)551-556; Lu et al., Anesthesia and Analgesia 98:414-419(2004); and Yeomans et al., Molec. Therapy (2004) 9:24-29.

### SUMMARY OF THE INVENTION

Methods and compositions are provided for determining whether nucleic acid sequence of a sensory neuronal cell is involved in sensory function, by being involved in a cell processes critical to sensory function, such as signal transduction, signal conduction, second messenger activation, gene expression, and signal transmission. In practicing the subject methods, a neuronal cell selective vector including a modulating domain for a neuronal cell nucleic acid sequence with is administered to an animal. Sensory function in whole animals and in harvested neuronal cells is then evaluated to determine whether the nucleic acid sequence is involved in sensory function. Also provided are compositions, kits, and systems for practicing the subject methods.

### FEATURES OF THE INVENTION

One feature of the invention provides methods for determining whether an nucleic acid sequence (Which may be an endogenous or non-endogenous (e.g., novel) sequence) of a neuronal cell is involved in sensory function, by administering to an animal a neuronal cell selective vector comprising a modulating domain for the nucleic acid sequence, and optionally a domain encoding a directly detectable product; harvesting a neuronal cell from the animal that comprises the vector; and then evaluating conduction in the harvested cell to determine whether the endogenous nucleic acid sequence is involved in sensory function. In such methods, the endogenous nucleic acid sequence may encode a product and the neuronal cell may be a nociceptor (pain sensing neuron). In addition, the animal used in such methods may be a mammal, such as a rodent. In such methods, the harvesting may comprise selecting neuronal cells from the animal that are positive for the directly detectable product.

Furthermore, in such methods, the modulating domain may reduce expression of the target nucleic acid sequence (i.e., the nucleic acid sequence being assayed), or it may increase expression of the target nucleic acid sequence or it may induce the expression of the target nucleic acid sequence. The modulating domain may encode an antisense product, a RNAi product, or at least one copy of the target nucleic acid. The directly detectable product, in such methods, may be a fluorescent protein. In addition, the neuronal cell selective vector may be a herpes simplex virus vector, such as a Herpes Simplex Virus Type 1 vector.

Another feature of the invention provides compositions that include a neuronal cell selective vector comprising a neuronal cell target nucleic acid modulating domain, and optionally a domain encoding a directly detectable product. In such compositions, the neuronal cell nucleic acid sequence may encode a product and the neuronal cell may be a nociceptor. Such a modulating domain may encode an antisense product, a RNAi product, or at least one copy of the target nucleic acid. Furthermore, the modulating domain may reduce expression of the target sequence or it may increase expression of the target nucleic acid sequence or induce the expression of the target nucleic acid sequence. Such a neuronal cell selective vector may be a herpes simplex virus vector, such as a Herpes Simplex Virus Type 1 vector. In addition, in such compositions the directly detectable product may be a fluorescent protein.

Yet another feature of the invention provides kits and systems which comprise a neuronal cell specific vector comprising a neuronal cell target nucleic acid modulating domain and optionally a domain encoding a directly detectable product, or a pro-vector thereof, and instructions for using the vector to determine whether a target nucleic acid sequence of a neuronal cell is involved in sensory function. The kits and systems of the invention may also comprise a neuronal cell sensory function testing element, a neuronal cell harvesting element, and an animal.

### DETAILED DESCRIPTION OF THE INVENTION

Methods and compositions are provided for determining whether target nucleic acid sequence of a neuronal cell is involved in sensory function. In practicing the subject methods, a neuronal cell selective vector including a modulating domain for a neuronal cell target nucleic acid sequence is administered to an animal. Sensory function in harvested neuronal cells is then evaluated to determine whether the endogenous nucleic acid sequence is involved in sensory function. Also provided are compositions, kits, and systems for practicing the subject methods.

Before the present invention is described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a neuronal cell” includes a plurality of such neuronal cells and reference to “the vector” includes reference to one or more vectors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the subject invention, the methods will be described first, followed by a review of representative applications in which the methods find use, as well as a review of representative kits and systems that find use in practicing the subject methods.

Methods

As summarized above, the subject invention provides methods and compositions for determining whether a target nucleic acid sequence of a neuronal cell is involved in sensory function, e.g., by modulating the expression of the nucleic acid sequence product. By “involved” it is meant to be connected by participation or association. A given target nucleic acid is considered to be involved in sensory function if it is involved in a cell process critical to sensory function, such as signal transduction, signal conduction, second messenger activation, gene expression, and signal transmission. By “signal transduction” it is meant the conversion of, for example, light, pressure, and chemicals into nerve impulses by neuronal cells. The term modulation includes both decreasing (inhibiting) and enhancing expression.

Accordingly, modulation may be in the form of either inhibiting expression of the target nucleic acid sequence, or enhancing the expression of the target nucleic acid sequence or inducing the expression of a target nucleic acid sequence. By inhibiting expression is meant at least decreasing the extent of expression in the neuronal cell of the endogenous nucleic acid sequence, by at least about 10 to 20% as compared to a control, or substantially if not completely stopping expression, such that the product is not present in the neuronal cell. By enhancing expression is meant at least increasing the extent of expression in the neuronal cell of the endogenous nucleic acid sequence, at least about 10 to about 20% as compared to a control, or where expression is very low in the neuronal cell, enhancing means at least causing at least detectable expression in the neuronal cell. By inducing expression of a target nucleic acid sequence is meant the induction of any amount of target nucleic acid sequence.

The subject methods may be used to modulate expression in sensory neuronal cells in a variety of different types of subjects. Generally the subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be rodents.

Expression is modulated in a subject animal according to the subject methods by administering to the subject animal a neuronal cell selective vector comprising a modulating domain for a target nucleic acid sequence, optionally with (i.e., with or without) a domain encoding a directly detectable product. The term “neuronal cell” refers to any of the signal conducting cells of the nervous system, where in many embodiments the specific neuronal cells of interest are sensory neurons, such as nociceptors. Modulating expression includes both increasing and decreasing the expression level of the target neuronal cell nucleic acid sequence.

As indicated above, the modulating domain for the target nucleic acid sequence may enhance or inhibit expression of the target neuronal cell nucleic acid sequence. The modulating domain for the endogenous nucleic acid sequence may be a variety of different polynucleotide compositions, e.g., coding sequences, antisense compositions, siRNA compositions, etc.

In some embodiments, the modulating domain for the target nucleic acid sequence administered to the host is a polynucleotide or nucleic acid composition. The nucleic acid composition may be coding sequences, e.g., genes, gene fragments etc., which may be present in expression vectors, where such vectors generally have convenient restriction sites located near the promoter sequence to provide for the insertion of nucleic acid sequences. Transcription cassettes may be prepared that include a transcription initiation region, the target gene or fragment thereof, and a transcriptional termination region. The transcription cassettes may be introduced into a variety of vectors, e.g., plasmid; retrovirus, e.g., lentivirus; adenovirus; and the like, where the vectors are able to transiently or stably be maintained in the cells, usually for a period of at least about one day, more usually for a period of at least about several days to several weeks.

The modulating domain for the endogenous nucleic acid sequence may be nucleic acid sequence form the subject animal or a homolog of nucleic acid sequence (or fragments thereof from other species, i.e., other animal species, where such homologs or proteins may be from a variety of different types of species, usually mammals, e.g., rodents, such as mice, rats; domestic animals, e.g. horse, cow, dog, cat; and primates, e.g., monkeys, baboons, humans etc. By homolog is meant a nucleic acid having at least about 35%, usually at least about 40% and more usually at least about 60% sequence identity to the specific human nucleic acids as identified above, where sequence identity is as measured by the BLAST Compare Two Sequences program available on the NCBI website using default settings.

In other embodiments of the invention, the modulating domain for the endogenous nucleic acid sequence is an agent that modulates, and generally decreases or down regulates, expression of the target nucleic acid sequence in the subject animal. Antisense molecules can be used to down-regulate expression of a gene in cells. The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g., by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate neuronal call specific vector, where the transcriptional initiation is oriented such that an antisense strand is produced as an RNA molecule. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the neuronal cell endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

As an alternative to anti-sense inhibitors, catalytic nucleic acid compounds, e.g. ribozymes, anti-sense conjugates, etc. may be used to inhibit expression of endogenous neuronal cell nucleic acid sequence. Ribozymes may be encoded on a neuronal cell selective vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridyl Cu(II), capable of mediating mRNA hydrolysis are described in Bashkin et al. (1995), Appl. Biochem. Biotechnol. 54:43-56.

Alternatively, gene expression can be modified by gene silencing using double-strand RNA (Sharp (1999) Genes and Development 13:139-141). RNAi, otherwise known as double-stranded RNA interference (dsRNAi) or small interfering RNA (siRNA), has been extensively documented in the nematode C. elegans (Fire, A., et al, Nature, 391, 806-811, 1998). In such embodiments the RNAi agent is a transcriptional template of the interfering ribonucleic acid. In these embodiments, the transcriptional template is typically a DNA that encodes the interfering ribonucleic acid. The DNA is present in a neuronal cell selective vector, where a variety of different vectors are known in the art and can be modified to be selective for neuronal cells, e.g., a viral vector (such as a HSV vector), etc.

In such embodiments, an effective amount of an RNAi agent is introduced into the target neuronal cell to modulate expression of a target nucleic acid sequence in a desirable manner, e.g., to achieve the desired reduction in target neuronal cell nucleic acid sequence expression. The resulting expressed RNAi are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, 23 bp are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

As indicated above, the modulating domain for the endogenous nucleic acid sequence is administered to the subject animal by a neuronal cell selective vector. For example, a subject polynucleotide can be administered to neuronal cells using either a “non-viral delivery vehicle” or a “viral delivery vehicle,” wherein such delivery vehicles have been modified to be neuronal cell selective. By “neuronal cell selective” is meant that the delivery vehicle is selective for neuronal cells over other cell types.

A subject delivery vehicle may be modified to be neuronal cell selective using any means known in the art. For example, the delivery vehicle may be modified to include a targeting ligand that enables the delivery vehicle to be directed selectively to a target receptor on neuronal cells. By “targeting agent” is meant a chemical structure, e.g. antibody, which binds with a degree of specificity to a targeting receptor that is enriched at a target cell compared to at a non-target cell. By “target receptor” is meant a chemical structure at the target cells that binds with a useful degree of specificity to a targeting ligand wherein said target receptor is present in increased amounts at the target cells compared to at some non-target cells.

“Non-viral delivery vehicle” (also referred to herein as “non-viral vector”) as used herein is meant to include chemical formulations containing naked or condensed polynucleotides (e.g, a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel et al. 1992 Am. J. Respir. Cell Mol. Biol. 6:247-52)). Thus “non-viral delivery vehicle” can include vectors composed of polynucleotides plus viral particles where the viral particles do not contain the polynucleotide of interest. “Non-viral delivery vehicles” include bacterial plasmids, viral genomes or portions thereof, wherein the polynucleotide to be delivered is not encapsulated or contained within a viral particle, and constructs comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Felgner et al (U.S. Pat. Nos. 5,264,618 and 5,459,127; PNAS 84:7413-7417, 1987; Annals N.Y. Acad. Sci. 772:126-139, 1995); they may also consist of neutral or negatively charged phospholipids or mixtures thereof including artificial viral envelopes as disclosed by Schreier et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI) (see Boussif et al., PNAS 92:7297-7301, 1995) which molecules can also serve as condensing agents. These carriers may be dissolved, dispersed or suspended in a dispersion liquid such as water, ethanol, saline solutions and mixtures thereof. A wide variety of synthetic polymers are known in the art and can be used.

“Non-viral delivery vehicles” further include bacteria. The use of various bacteria as delivery vehicles for polynucleotides has been described. Any known bacterium can be used as a delivery vehicle, including, but not limited to non-pathogenic strains of Staphylococcus, Salmonella, and the like.

The polynucleotide to be delivered can be formulated as a DNA- or RNA-liposome complex formulation. Such complexes comprise a mixture of lipids which bind to genetic material (DNA or RNA) by means of cationic charge (electrostatic interaction). Cationic liposomes which may be used in the present invention include 3β-[N-(N′,N′-dimethyl-aminoethane)-carbamoyl]-cholesterol (DC-Chol), 1,2-bis(oleoyloxy-3-trimethylammonio-propane (DOTAP) (see, for example, WO 98/07408), lysinylphosphatidylethanolamine (L-PE), lipopolyamines such as lipospermine, N-(2-hydroxyethyl)-N,N-dimethyl-2,3-bis(dodecyloxy)-1-propanaminium bromide, dimethyl dioctadecyl ammonium bromide (DDAB), dioleoylphosphatidyl ethanolamine (DOPE), dioleoylphosphatidyl choline (DOPC), N(1,2,3-dioleyloxy) propyl-N,N,N-triethylammonium (DOTMA), DOSPA, DMRIE, GL-67, GL-89, Lipofectin, and Lipofectamine (Thiery et al. (1997) Gene Ther. 4:226-237; Felgner et al., Annals N.Y. Acad. Sci. 772:126-139, 1995; Eastman et al., Hum. Gene Ther. 8:765-773, 1997). Polynucleotide/lipid formulations described in U.S. Pat. No. 5,858,784 can also be used in the methods described herein. Many of these lipids are commercially available from, for example, Boehringer-Mannheim, and Avanti Polar Lipids (Birmingham, Ala.). Also encompassed are the cationic phospholipids found in U.S. Pat. Nos. 5,264,618, 5,223,263, and 5,459,127. Other suitable phospholipids which may be used include phosphatidylcholine, phosphatidylserine, phosphatidylethanolamine, sphingomyelin, phosphatidylinositol, and the like. Cholesterol may also be included.

A subject polynucleotide can also be associated with viral delivery vehicles. As used herein, a “viral delivery vehicle” intends that the polynucleotide to be delivered is encapsidated in a viral particle.

Numerous viral genomes useful in in vivo transformation and gene therapy are known in the art, or can be readily constructed given the skill and knowledge in the art. Included are replication competent, replication deficient, and replication conditional viruses. Viral vectors include adenovirus, mumps virus, a retrovirus, adeno-associated virus, herpes simplex virus (HSV), cytomegalovirus (CMV), vaccinia virus, and poliovirus, and non-replicative mutants/variants of the foregoing. In some embodiments, a replication-deficient virus is capable of infecting slowly replicating and/or terminally differentiated cells, since the respiratory tract is primarily composed of these cell types. For example, adenovirus efficiently infects slowly replicating and/or terminally differentiated cells. In some embodiments, the viral genome itself, or a protein on the viral surface, is specific or substantially specific for cells of the targeted cell. A viral genome can be designed to be target cell-specific by inclusion of cell type-specific promoters and/or enhancers operably linked to a gene(s) essential for viral replication.

Where a replication-deficient virus is used as the viral genome, the production of virus particles containing either DNA or RNA corresponding to the polynucleotide of interest can be produced by introducing the viral construct into a recombinant cell line which provides the missing components essential for viral replication and/or production. Preferably, transformation of the recombinant cell line with the recombinant viral genome will not result in production of replication-competent viruses, e.g., by homologous recombination of the viral sequences of the recombinant cell line into the introduced viral genome. Methods for production of replication-deficient viral particles containing a nucleic acid of interest are well known in the art and are described in, for example, Rosenfeld et al., Science 252:431-434, 1991; Rosenfeld et al., Cell 68:143-155, 1992 (adenovirus); U.S. Pat. No. 5,139,941 (adeno-associated virus); U.S. Pat. No. 4,861,719 (retrovirus); and U.S. Pat. No. 5,356,806 (vaccinia virus). Methods and materials for manipulation of the mumps virus genome, characterization of mumps virus genes responsible for viral fusion and viral replication, and the structure and sequence of the mumps viral genome are described in Tanabayashi et al., J. Virol. 67:2928-2931, 1993; Takeuchi et al., Archiv. Virol., 128:177-183, 1993; Tanabayashi et al., Virol. 187:801-804, 1992; Kawano et al., Virol., 179:857-861, 1990; Elango et al., J. Gen. Virol. 69:2893-28900, 1988.

In a representative embodiment, the neuronal cell specific vector is a recombinant herpes simplex virus, such as HSV-1. The recombinant herpes simplex virus may be debilitated for growth via non-silent insertion, substitution, or deletion of a nucleotide sequence in at least one non-essential gene of the herpes simplex virus. In a related embodiment, the recombinant herpes simplex virus may further comprise a non-silent insertion, substitution, or deletion of a nucleotide sequence in at least one essential gene of the herpes simplex virus. In one embodiment, the herpes simplex virus lacks one expressible γ_{L}34.5 gene, a non-essential gene. In a further embodiment, the recombinant herpes simplex lacks both expressible γ_{L}34.5 genes.

As noted above the neuronal cell specific vector may also include an RNA stabilizing element embodiment. By “RNA stabilizing element” it is meant an RNA export element which mediates efficient transport of RNA from the nucleus to the cytoplasm. In a preferred embodiment the RNA stabilizing element is Woodchuck Posttranslational Regulatory Element, described in U.S. Pat. No. 6,284,469, the disclosure of which is herein incorporated by reference.

As noted above, the neuronal cell specific vector may also include a domain encoding a directly detetctable marker. The directly detectable marker may be fluorescent, luminescent, radioactive, etc. In certain embodiments, the directly detetctable marker is a fluorescent protein. As used herein, the term “fluorescent protein” refers to any protein capable of fluorescence when excited with appropriate electromagnetic radiation. This includes fluorescent proteins whose amino acid sequences are either naturally occurring or engineered (i.e., mutants or analogs). Fluorescent proteins of interest include, but are not limited to: (1) the Aequoria victoria green fluorescent proteins and variants thereof, such as those described in U.S. Pat. Nos.: 6,066,476; 6,020,192; 5,985,577; 5,976,796; 5,968,750; 5,968,738; 5,958,713; 5,919,445; 5,874,304; and 5,491,084; the disclosures of which are herein incorporated by reference, as well as International Patent Publications: WO 00/46233; WO 99/49019; and DE 197 18 640; and the Anthozoa derived fluorescent proteins, including but no limited to: (1) amFP485, cFP484, zFP506, zFP540, drFP585, dsFP484, asFP600, dgFP512, dmFP592, as disclosed in published U.S. application US-2002-0197676-A1; the disclosure of which is herein incorporated by reference

In practicing the subject methods, the neuronal cell selective vector is administered to the subject animal using any convenient means.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles. An agent of the invention may also be administered topically. Typically, topical compositions are prepared as liquid solutions of suspensions, solid forms suitable for soution in, or suspension in, liquid vehicles prior to topical application may also be prepared. The topical preparation may also be emulsified or the active ingredient encapsulated in liposome vehicles.

Following administration of the neuronal cell specific vector, an effective amount of time is allowed to pass. By “effective amount of time” is meant an amount of time necessary to enable the vector to enter the neuronal cells and allow the modulating domain for the endogenous nucleic acid sequence and the domain encoding the directly detectable product, if present, to be expressed. The amount of time allowed to pass will range from about 1 day to about 120 days, such as from about 2 days to about 30 days, and including from about 3 days to about 21 days.

In practicing the subject methods, the animal and the neuronal cells of the animal including the neuronal cell specific vector may then be evaluated to determine whether the endogenous nucleic acid sequence is involved in sensory function.

In some embodiments, the animal which includes the neuronal cell specific vector is evaluated to determine what effect the modulating domain for the endogenous nucleic acid sequence has on sensory function. Such evaluation methods may include any behavioral testing methods known in the art.

Behavioral testing of the effect a modulating domain of an endogenous nucleic acid sequence has on neuronal sensory function may be performed by a foot withdrawal test, as described in Yeomans et al., Pain, 68:133-140, 1996; Yeomans and Proudfit, Pain, 68:141-150, 1996. In such a testing method, the latency of withdrawal of the foot of the animal in response to stimulation is measured. Such stimulation may be in the form of pain, such as by local exposure to a heat source. If the modulating domain of an endogenous nucleic acid sequence has an effect of decreasing the amplitude of neuronal sensory function then the sensation of pain is decreased. Therefore, the latency of withdrawal in response to pain stimulation will be higher then that of a control animal. Alternatively, if a the modulating domain of an endogenous nucleic acid sequence has an effect of increasing the amplitude of neuronal cell sensory function then the sensation of pain is increased. Therefore, the latency of withdrawal in response to pain stimulation will be lower then that of a control animal. Such latency measurements may be taken immediately following local stimulation. Additional measurements may be taken anywhere from about 10 sec to about 3 hours after local stimulation.

In addition, the response latency to inflammation may also be evaluated following local induction of the inflammatory response by local administration of an inflammatory agent, such as Complete Freund's Adjuvant (CFA). This treatment typically increases pain sensitivity and so decreases response latencies. If the modulating domain of an endogenous nucleic acid sequence has an effect of decreasing neuronal sensory function then the response latency after inflammation is increased. Alternatively, if a the modulating domain of an endogenous nucleic acid sequence has an effect of increasing neuronal sensory function, then the response latency after inflammation is decreased. Such latency measurements may be taken immediately following local stimulation. Additional measurements may be taken anywhere from about 1 hr to about 30 days after local stimulation.

In further practicing the subject methods, the effect of the modulating domain of a target nucleic acid on neuronal sensory function may also be evaluated by examining the neuronal cells, such as dorsal root ganglion cells, that include the neuronal cell selective vector. Such cells may be harvested from the animal and then evaluated to determine whether the target nucleic acid sequence is involved in sensory function. Such harvesting may be performed by any means known in the art and facilitated by the selection of neuronal cells exhibiting a positive signal for the directly detectable product. For example, harvesting of the neuronal cells that include the neuronal cell specific vector may be done by using a surgical scalpel to remove the dorsal root ganglion from the euthanized animal.

Such methods of evaluating the effect of the modulating domain for the endogenous nucleic acid sequence on neuronal cell function in harvested sensory neuronal cells that include the neuronal cell selective vector may include any procedure described in the art, e.g., biochemical and immunological.

In some embodiments patch clamp recordings of the harvested cells are conducted to evaluate the effect of the modulating domain for the target nucleic acid sequence on neuronal sensory function. Patch clamp recording methods, as described in Brock, Mathes and Gilly, J. Gen. Physiol., 118:113-133, 2001, are performed to investigate ion channel activity in the harvested neuronal cells containing the neuronal cell selective vector. For example, where the target nucleic acid sequence encodes an ion channel involved in sensory function, and the expression of the channel is decreased as a result if the presence of the modulating domain for the target nucleic acid sequence, then the recordings for the channel activity will be decreased accordingly, compared to control cells. Likewise, if the modulating domain increases expression for the target nucleic acid product, the recordings for the channel activity will be increased compared to control cells. Likewise, if the modulating domain induces expression of a target nucleic acid product, the recordings for the channel activity will be increased compared to control cells.

In other embodiments semi-quantitation of channel expression via western blot of extracts from the harvested cells are conducted to evaluate the effect of the modulating domain for the target nucleic acid sequence on neuronal sensory function. Semi-quantitation of channel expression via western blot, as described in Toledo-Aral et al., PNAS, 94:1527-1532, 1997, may be used to assess the ability of the modulating domain for the target nucleic acid in either increasing or decreasing the expression of the target nucleic acid sequence product. For example, where the target nucleic acid sequence encodes a channel involved in sensory function, expression levels of the channel may be evaluated using western blot by determining the level of expression in control neuronal cells and cells containing the neuronal cell specific vector.

In yet other embodiments immunohistochemical methods may also be used to examine harvested neuronal cells to evaluate the effect of the modulating domain for the target nucleic acid sequence on neuronal sensory function. Immunohistochemical methods may be used to assess the ability of the modulating domain for the target nucleic acid in either increasing or decreasing the expression of the protein or peptide end product of the nucleic acid sequence. Such methods are performed by chemically fixing harvested neuronal cells that contain the neuronal cell specific vector to slides and incubating the cells with primary antibodies to the target neuronal cell nucleic acid product and fluorescent secondary antibodies according to methods known in the art. Immunostaining of the endogenous neuronal cell nucleic acid product may then be assessing using methods known in the art, such as laser scanning confocal microscopy and using computer software to analyze captured images of the stained cells. For example, where the target nucleic acid sequence encodes a channel involved in sensory function, expression levels of the channel may be evaluated as witnessed by the immunostaining on the harvested cells containing the neuronal cell specific vector as compared to control cells.

Utility

The subject methods and compositions find use in a variety of different applications where it is desired to evaluate the impact of a target, e.g., an endogenous or novel (non-endogenous), nucleic acid sequence and/or its protein/peptide end-product on sensory neuronal cell function. As such, the subject methods and compositions find use in applications to produce a sensory neuronal cell selective knockout animal to study the role of neuronal cell target gene products in cell function.

Such neuronal cell animal models are useful in studying potential targets for candidate analgesic and other sensory modifying agents as well as evaluating the effectiveness of a candidate analgesic or other sensory modifying agent. For example, the neuronal cell selective knockout animal may be used in assays to evaluate the role the knocked out nucleic acid-sequence plays in sensory function by comparing sensory responses in the subject animal model to sensory responses in a non-modified animal. The information received from the studies is useful in determining potential targets for developing analgesic or other sensory modifying agents. In addition, such animal models are also useful in determining whether a candidate analgesic or other sensory modifying agent is effective at alleviating the phenotype caused by the increased or decreased nucleic acid product.

Another embodiment is the use of the subject methods and compositions to introduce a neuronal cell target nucleic acid in order to increase target nucleic acid product in the neuronal cell of an animal. Such neuronal cell animal models are also applicable in studying potential targets for candidate analgesic agents.

For example the animal models are useful in assays evaluating the effect a nucleic acid sequence has on neuronal cell sensory function by comparing sensory function in the subject animal model to that of a non-modified animal. The information received from the studies is useful in determining potential targets for developing analgesic or other sensory modifying agents. In addition, such animal models are also useful in determining whether a candidate analgesic agent or other sensory modifying is effective at altering the phenotype caused by the increased target nucleic acid product.

Kits

Also provided are kits that find use in practicing the subject methods, as described above. For example, in some embodiments, kits for practicing the subject methods may include a neuronal cell specific vector previously prepared to include a domain encoding a directly detectable product and a neuronal cell target nucleic acid sequence. Alternatively, in other embodiments, kits may include a neuronal cell specific pro-vector previously prepared to include the domain encoding a directly detectable product and a plasmid containing a cloning cassette ready for insertion of desired neuronal cell nucleic acid sequence. In yet other embodiments, kits may include a neuronal cell specific pro-vector, and a plasmid containing a cloning cassette ready for insertion of desired neuronal cell nucleic acid sequence. The kits for practicing the subject methods may also include neuronal sensory function testing element, a neuronal cell harvesting element, and an animal.

For example, the testing element of such kits may include a variety of locally administrable stimulatory agents, such as an inflammatory agent for conducting behavioral testing on the animal, such as foot withdrawal tests and inflammatory response latency tests. In addition, the testing element may also include chemicals, solutions, and buffers necessary to perform biochemical, neurophysiological, and immunological tests on harvested neuronal cells containing the neuronal cell selective vector. Such neurophysiological test may include patch clamp recordings of the harvested cells. Additionally, such immunological and biochemical tests may include semi-quantitation of channel expression via western blots and immunocytochemical tests of the harvested cells.

The neuronal cell harvesting element of such kits may include a surgical scalpel, other surgical instruments, and any solutions and buffers necessary to facilitate the harvesting of neuronal cells containing the neuronal cell selective vector from the euthanized animal.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems that find use in practicing the subject methods, as described above. For example, in some embodiments, systems for practicing the subject methods may include a neuronal cell selective vector including a neuronal cell target nucleic acid modulating domain with or without a domain encoding a directly detectable product, an animal, a neuronal cell harvesting element, and a sensory function testing element.

For example, the sensory function testing element of such systems may include a variety of locally administrable stimulatory agents, such as an inflammatory agent for conducting behavioral testing on the animal, such as foot withdrawal tests and inflammatory response latency tests. In addition, the testing element may also include chemicals, solutions, and buffers necessary to perform biochemical and immunological tests on harvested neuronal cells containing the neuronal cell selective vector. Such biochemical test may include patch clamp recordings of the harvested cells. Additionally, such immunological tests may include semi-quantitation of channel expression via western blots and immunocytochemical tests of the harvested cells.

The neuronal cell harvesting element of such systems may include a surgical scalpel and any solutions and buffers necessary to facilitate the harvesting of neuronal cells containing the neuronal cell selective vector from the euthanized animal.

The following examples are offered by way of illustration and not by way of limitation.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

I. Materials and Methods

A. Animals

All animal procedures were approved and followed guidelines of the Stanford University Administrative Panel on Laboratory Animal Care (A-PLAC). Foot depilation, scarification, and viral was performed following administration of˜330 mg tribromoethanol/kg body weight (i.p.). Testing of the foot-withdrawal response to noxious radiant heat with or without CFA injection was performed while animals are lightly anesthetized with tribromoethanol (200 mg/kg, i.p.). For euthanasia, mice were decapitated after asphyxiation with CO₂.

B. Virus Construction and Purification

A recombinant herpes simplex virus type I (HSV) vector designed to knockdown expression of the Naᵥ1.7 sodium channel, designated NGNav1.7 was constructed by standard techniques. Briefly, a shuttle cassette was constructed containing the cDNA for the full-length (rat) Naᵥ1.7 sodium channel inserted in antisense orientation relative to the hCMV immediate-early enhancer-promoter promoter. This construct provided transcription of an antisense RNA complementary to the Naᵥ1.7 mRNA and therefore reduction in expression of the Naᵥ1.7 protein in infected cells. This transcription cassette also contained a woodchuck hepatitis virus element (WPRE) to enhance RNA stability and was flanked by HSV DNA from the UL36 and UL37 genes.

This shuttle plasmid was then linearized and transfected with DNA from the NEGFP virus that had been digested with Spe I to cut the unique restriction site located at the terminus of the UL37 gene. This technique resulted in a high percentage of recombinants, similar to that found when a unique restriction site is introduced into HSV (Krisky et al., Gene Ther., 4:1120-5, 1997). Virus NEGFP is based on the non-neurovirulent KOS strain of HSV and expresses enhanced green fluorescent protein (EGFP) driven by the hCMV promoter. The EGFP expression cassette is inserted into the viral thymidine kinase gene, thus preventing replication in non-dividing cells such as neurons and reactivation from latency (Tenser et al., J. Virol., 63:2861-5, 1989). Virus NEGFP also contained a deletion of both copies of the γ_{L}34.5 gene to reduce neurotoxicity.

The complete rat sequence for Naᵥ 1.7 was used in these studies. However, as an alternative we also constructd a virus encoding the 309 bp N-terminal sequence of murine Naᵥ1.7, which is known, and which is critical to channel function (Lai et al., Methods Enzymol., 314:201-213, 2000). Virus stocks were produced by infection of Vero cells, isolated by centrifugation through 30% sucrose in PBS and stored in 10% sucrose in PBS at 80° C.

C. Virus Application

Mice were anesthetized by administering˜330 mg/kg tribromoethanol (i.p.). Hair was removed from the dorsal surface of both hindpaws by application of Nair® for 3-5 min followed by rinsing of the paws with water. The dorsal skin of each hindpaw was then scarified by light application of a motorized sanding drum. Either 5 μl of vehicle or suspensions either virus NGNav1.7 or control virus NEGFP containing 10⁷ plaque-forming units in 5 μl were then applied to the dorsal surface of the left hindpaw and gently distributed using the side of a disposable pipettor tip. After 10 minutes, when the virus suspension had absorbed/dried onto the foot, the animals were returned to their home cages.

D. Behavioral Testing

The effects of the vectors on thermonociception was assessed using the Aδ/C foot withdrawal test (Yeomans et al., 1996; Yeomans and Proudfit, 1996). Mice were lightly anesthetized with tribromoethanol (200 mg/kg). Three sets of withdrawal latency measurements were made when the dorsal hairy surface of the hindpaw was heated alternately at either a high (Aδ selective) or low (C selective) rate. Typical response latencies for these stimuli were 2.5-3 s for Aδ and 12-13 s for C fiber thermonociceptive responses. Maximal (cut-off) latencies were set at 6 and 20 s respectively to limit potential skin damage. Measurements were separated by 3-minute intervals to minimize sensitization/desensitization that can occur with repeated skin heating. Response latency measurements were made prior to viral application, then at 2 weeks after application.

After the final test, rats were administered a 10 μl subcutaneous injection of saline or CFA (Mycobacterium tuberculosis; Sigma) in saline to induce a local inflammatory response and increased Naᵥ1.7 expression (Gould et al, Brian Res. 824:296-9, 1999). Response latencies were then measured at 1 and 2 hr after CFA application. The mice were then allowed to recover in their home cages. One week later, the mice were once again lightly anesthetized and latency measurements were again taken to determine whether either virus changed responsiveness to CFA application. Mice were then be deeply anesthetized with the addition of more tribromoethanol, perfused with 4% paraformaldehyde/picric acid, and (DRG were removed in preparation for immunohistochemical measurements of Naᵥ1.7 levels (see below).

E. DRG Dissociation

After final behavioral testing, mice were euthanized with CO₂,and L3-L5 DRGs were removed from each side. Some DRGs were prepared for immunoassay of Naᵥ1.7 levels (see below). Others were treated in Tryrode's (in mM: 140 NaCl, 4 KCl, 2 CaCl₂, 2 MgCl₂, 10 glucose, 10 HEPES (pH7.4)) with protease (5 mg/ml dispase) plus 2 mg/ml collagenase (Sigma Type 1) at 35° C. with gentle shaking for 60 min. After decanting most of the fluid, cells were released by trituration. Enzyme solution was added back for 15 min, and cells were pelleted by centrifugation at 500 rpm for 30 sec. After replacing the enzyme solution with Tyrode, a final centrifugation was carried out. Cells were plated onto 5 mm diameter glass coverslips coated with poly-l-lysine in 35 mm culture dishes and cultured in DMEM plus 10% fetal bovine serum with 5% CO at 37° C.

F. Patch Clamp Recordings

Conventional whole-cell patch clamp methods were used (Brock, Mathes and Gilly, J. Gen. Physiol., 118:113-133, 2001). Neurons prepared as described sprout processes in vitro, but most remain suitable for recording for 24 hours. Whole-cell recordings were therefore limited to this period, and only cells without processes were studied. External solution minimized calcium ion currents and contained (in mM) 140 NaCl, 0.5 CaCl₂, 10 MgCl₂, 10 HEPES (pH7.4). Internal solution eliminated potassium ion currents and contained (in mM) 40 NaCl, 60 NMG-F (or CsF), 30 NMG-Cl (or CsCl), 20 tetraethylammonium (TEA) Cl, 1 MgCl₂, 10 EGTA, 10 HEPES (pH 7.4).

G. Semi-quantitation of Channel Expression via Western Blot of DRG Extracts

The effectiveness of repression of endogenous channel synthesis via antisense vectors was assessed using western blot analysis (see Toledo-Aral et al., PNAS, 94:1527-1532, 1997). Extracts of NGNav1.7 transfected and NEGFP (control)-transfected DRG or DRGs from vehicle treated mice were subjected to SDS-PAGE and blotted onto nitrocellulose, and the blots were developed using NaCh isoform-specific antibodies and chemiluminescence reagents (Pierce SuperSignal FemtoWest). Blots were imaged using a Kodak 440i cooled CCD based imager, which is nearly as sensitive as film but provides a linear response over about 5 orders of magnitude of intensity ranges.

With this method we were able to readily detect a femtomole of channel protein on a blot, and a single DRG produces well-resolved bands on a western blot with a few minutes of exposure time. However, where the transfection efficiency was low, then the few transfected cells did not yield detectable differences from the control ganglion. In this case, we enzymatically dissociated the ganglia and then used FACS to isolate GFP-expressing cells in both channel antisense transgene, NGNav1.7 and NEGFP transfected ganglia. Based on a electrophysiologically-determined estimate of at least 10,000 channels on the cell surface membrane and a cytoplasmic organelle (ER plus Golgi) component of about 100 times this amount (e.g. see Thornhill and Levinson, Methods Enzymol., 207:659-670, 1992; Ukomadu et al., Neuron, 8:663-676, 1992), a few hundred cells from either NEGFP or NGNav1.7 transfected mice were gathered for the comparison. These same methods were applied to DRGs from animals that had received subcutaneous complete Freund's adjuvant (CFA) injections one week prior to cell harvesting, in order to determine the effects of viral application on the increase in Naᵥ1.7 protein levels.

H. Immunocytochemical Methods

As an adjunct, suppression of Naᵥ1.7 was also evaluated both in DRG and in nerve processes using immunocytochemical methods. For DRGs, animals were fixed by perfusion with 4% paraformaldehyde/picric acid, ganglia harvested, embedded in cryoprotectant, and cryosectioned onto bonding SuperFrost Plus slides (15-30μ sections). Sciatic nerves were dissected from the thigh, cut into 1 cm segments, and then teased onto slides into thin spreads. Specimens affixed to slides were then incubated with primary and fluorescent secondary antibodies according to standard protocols. Immunostaining was assessed using laser scanning confocal microscopy (Nikon PCM-2000), and images were acquired and analyzed using SimplePCI software (Compix, Inc.). For DRG, differences in Naᵥ1.7 abundance due to HSV-transfection were determined from the analysis of Naᵥ1.7 immunofluorescence intensity of GFP-positive neurons of ganglia of animals infected with virus NGNav1.7 versus GFP-positive cell profiles of animals that were infected with the control NEGFP virus. Similar comparisons were done on cell bodies of DRGs from non-transfected animals. Background fluorescence as determined from peptide antigen preblocked antibody controls were subtracted from values in experimental DRGs before such comparisons. For nerve spreads, immunofluorescence intensity comparisons were made on 1μ optical sections of unmyelinated fiber fascicles. These same methods were also applied to tissues from animals that had received subcutaneous CFA injections one week prior to removing DRGs and sciatic nerves, in order to determine immunocytochemically, whether the antisense virus diminished the expected robust increase in Naᵥ1.7 protein levels.

II. Results

A. Overview

In mammals, Na channels arise from 12 or more Naᵥ genes (Waxman et al., Novartis Found. Symp., 241:34-51, 2002). These Na channel “isoforms” exhibit functional differences (kinetics, voltage-dependence etc), subcellular localization differences (axons vs. nerve endings etc), and pharmacological differences (e.g. sensitivity to anti-arrhythmic and anti-seizure drugs) that affect how channels operate in normal and abnormal situations. Different Na channels also appear to be involved in different physiologic functions. Thus, particular Na channel isoforms appear to be critical in mechanisms underlying pain and have become an increasingly important target in pharmaceutical drug development programs. In addition, the expression of different Na channels is plastic.

Maladaptive changes in Na channel gene expression, and consequently Na currents, occur in some neuropathic states. For example, cutting peripheral nerves induces an upregulation of some Na channels, and a downregulation of others (Coward et al., Neuroreport, 12:495-500, 2001; Leffler et al., J. Neurophysiol., 88(2):650-658, 2002). The resultant changes in sodium flux result in prolonged hyperexcitability of sensory neurons, a condition which likely is critical to some kinds of chronic pain (Waxman et al., 2002; Baker and Wood, Trends Pharmacol. Sci., 22:27-31, 2001). Because Na channels are so diverse, and because channel expression is so plastic, it has been difficult to assess which channels are critical to different pain states. The present invention is a novel approach to examining this question. By using neuronal specific viral vector encoding antisense sequences to specific Na channel, combined with behavioral, immunochemical, and electrophysiological techniques, it is possible to study a channel's contribution and role in some pain states.

We have previously used herpes simplex viruses as vectors to alter the genome of primary afferent nociceptors (Wilson et al., I, 96:3211-3216, 1999; Wilson and Yeomans, Curr. Rev. Pain, 4:445-50, 2000). Herpes viruses have unique features that make them particularly useful for this purpose, including a selective neurotropism for sensory neurons, long-term residence in host neurons, and the capacity to carry very large transgenes without affecting the capacity of the virus to infect the cells. Thus, we have induced the expression of novel transgenes in primary afferent neurons, demonstrated full processing of the gene product, and a functional effect of this transfection (analgesia). In addition however, we have also demonstrated that we can selectively knock down endogenous genes in nociceptors by inserting transgenes into the virus in antisense orientation relative to the cytomegaloviral promotor (Wilson et al., 2000). Using this technique, we have knocked-down the peptide neurtotransmitter CGRP in spinal cord nociceptor terminals, as well as neurotransmitter receptors, and acid sensing ion channels.

The effects of topical application, to mouse hindpaw skin, of a virus encoding the gene for EGFP, a virus encoding both EGFP and, in antisense orientation, the gene for rat Naᵥ1.7, or vehicle was examined behaviorally, immunohistochemically, and electrophysiologically. Baseline behavioral thermonociceptive responses were measured in each of 3 groups of 40 male, Swiss-Webster mice. Thereafter, a solution of one of the 2 viruses, or vehicle was applied to one foot of each mouse. Two weeks later, behavioral thermonociceptive responses were remeasured. At this point, one-half of each group of mice was euthanized. One half of each of these subgroups (¼ of each starting group) had their DRGs removed and those cells which have been transfected, or similarly sized cells from non-infected animals were electrophysiologically characterized in terms of sodium ionic flux. The DRGs of the 10 mice that made up the other half of each subgroup were removed and examined immunochemically to determine the level of Naᵥ1.7 expression in each group. The other half of each of the original group (20 mice each) received a subcutaneous injection of Complete Freund's Adjuvent (CFA) and were retested for behavioral responsivity. A week later, the thermonociceptive responsiveness of these same animals was tested again to determine whether the CFA treatment has induced a chronic thermal hyperalgesia. At that time the mice were euthanized. As above, one half of each subgroup had DRGs removed for electrophysiological characterization and the other 10 mice had DRG nerves removed for immunochemical measurement of Naᵥ1.7 levels.

B. Behavioral testing

Application of either virus did not affect behavioral paw withdrawal responses in mice that had not received an injection of inflammatory adjuvent, indicating a lack of effect on basal pain responsivity. On the other hand, while animals that had received the control virus showed a significant increase in pain responsivity (hyperalgesia) after induction of inflammation, mice that had received the Nav1.7 antisense virus did not become hyperalgesic. These results indicate that a block of Nav1.7 expression increase after inflammation prevents the hyperalgesia that normally accompanies inflammation.

C. Immunohistochemical

Consistent with our previous findings, induction of inflammation by local injection of CFA produced a robust increase in Nav1.7 protein levels in dorsal root ganglia neurons. In animals upon which the Nav1.7 antisense vector was applied, this increase was largely prevented. On the other hand, application of the control vector had no effect on the increase in Nav1.7 levels seen after the induction of peripheral inflammation.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of determining whether a target nucleic acid sequence of a sensory neuronal cell is involved in sensory function, said method comprising:
(a) administering to an animal a neuronal cell selective vector comprising a modulating domain for said target nucleic acid sequence;
(b) harvesting a neuronal cell that comprises said vector from said animal; and
(c) evaluating conduction in said harvested cell to determine whether said endogenous nucleic acid sequence is involved in sensory function.

2. The method according to claim 1, wherein said target nucleic acid sequence encodes a product.

3. The method according to claim 2, wherein said modulating domain at least reduces expression of said target nucleic acid sequence.

4. The method according to claim 3, wherein said modulating domain encodes an antisense product.

5. The method according to claim 3, wherein said modulating domain encodes an RNAi product.

6. The method according to claim 2, wherein said modulating domain increases expression of said target nucleic acid sequence.

7. The method according to claim 2, wherein said modulating domain induces expression of said target nucleic acid sequence.

8. The method according to claim 7, wherein said modulating domain includes at least one copy of said target nucleic acid.

9. The method according to claim 1, wherein said neuronal cell is a nociceptor.

10. The method according to claim 1, wherein said animal is a mammal.

11. The method according to claim 9, wherein said mammal is rodent.

12. The method according to claim 1, wherein said vector is a herpes simplex-virus vector.

13. The method according to claim 12, wherein said herpes simplex virus vector is Herpes Simplex Virus Type 1 vector.

14. The method according to claim 1, wherein said vector further comprises a sequence encoding a directly detectable product.

15. The method according to claim 14, wherein said directly detectable product is a fluorescent protein.

16. The method according to claim 14, wherein said harvesting comprises selecting neuronal cells from said animal that are positive for said directly detectable product.

17. A neuronal cell selective vector comprising a neuronal cell target nucleic acid modulating domain and a domain encoding a directly detectable product.

18. The vector according to claim 17, wherein said modulating domain at least reduces expression of said target nucleic acid sequence.

19. The vector according to claim 18, wherein said modulating domain encodes an antisense product.

20. The vector according to claim 18, wherein said modulating domain encodes an RNAi product.

21. The vector according to claim 17, wherein said modulating domain increases expression of said target nucleic acid sequence.

22. The vector according to claim 21, wherein said modulating domain includes at least one copy of said target nucleic acid.

23. The vector according to claim 17, wherein said neuronal cell is a nociceptor.

24. The vector according to claim 17, wherein said vector is a herpes simplex virus vector.

25. The vector according to claim 24, wherein said herpes simplex virus vector is Herpes Simplex Virus Type 1 vector.

26. The vector according to claim 17, wherein said directly detectable product is a fluorescent protein.

27. A kit comprising:
a vector according to claim 17 or a provector thereof; and
instructions for using said vector to determine whether a target nucleic acid sequence of a sensory neuronal cell is involved in sensory function.

28. The kit according to claim 27, wherein said kit further comprises a neuronal sensory function testing element.

29. The kit according to claim 27, wherein said kit further comprises a neuronal cell harvesting element.

30. A system comprising:
(a) a vector according to claim 17;
(b) an animal;
(c) a sensory neuronal cell harvesting element; and
(d) a neuronal sensory function testing element.
